SEQUENCE LISTING (6 sequences)

SEQ ID NO: 1 - DNA, 1979 bp, Oryzias latipes
  CDS (184)..(1911)
cttccttctgtgaagttatgcctagtttctggactctttgctcagctatgcaataagaaagaggattaacggagccgagcagcaggaatttggcctttcatccatcacctggttcccttaaagaaaatactcttcctctctttcctttctttctttctttctttcttctaccctctttccagcatgaccctgctgtcagaggaccagtctgccaggccccagccctgtctgctgtcagaacctgacaaacacatcagcacttctttagaccagtacagaccagactacactaaagaggactacatggacagtgtggagacctcagtgtttgggactgtggagccccctcgacgttctcgtggtcgcctgatcctccacagtatggttatgtttggtcgggagttctgctatgcggtggaagcggcatttgtcacaccggtgcttctcagcgtaggtctcccccgcagtctctacagtctggtgtggctgatcagccccatcctgggctttctgcttcagcccatcatcggctcagcaagtgactactgccggtcttcctggggaaggaggaggccctacatccttgtcctcggtatcctcatgctggtggggctcagcatgtttctcaatggagatgcagtggtctcagagctcgtcagtgacaggtcatccagaagtacgtgggccatcgtggtggtcatgtttggggttgtgctgttcgactttgctgcagatttcatcgacgggccaatcaaggcctacctgtttgatgtgtgctcataccaagacaaggaaagaggtcttcattatcacgctttattcacaggactgggcggagcttgtggttacctggttggagcaatggactggggtcactctgtattggggcgcctcctgggctctgagtaccaagtgatctacttcttctctgcactcacttggggcgtctttctcatcgtgcacctcttcagtattccagagaaacctctggccaaagtgccatctgaatcctcagcttccagtgcgctccgtttactaggcccccacagcaatggctacggagcactggggaaggagcctgtctctcctgtcatcccaacatccagccccgagatcaggccaaggtcctactcggcgcttggagaaagacccagatctttctctgctctcggtgaagccaactctgtgacttcaagtgccaaacagccaattaaggaggaccagaagaagatgacgttcaggtctttgatgaaagctatcttcaacatgccaaaccactaccgcttcctgtgcatcagccacctgctgggatgggcagcttttctctgcaacatgctcttcttcactgacttcatgggacagattgtttataggggaaatccttatgcagaacacaactccacggcttacatcacgtatgagagaggagttgaagttggctgctggggactgtgtattaatgcagtttcctctgctctctactcgtacgtgcagcgcttcctccttccatacattggcctgaagggattatacttcatgggctactttgtgttcggcatgggtaccagtctaattggcctcttcccagaagtcatagccacgctcatcctctgcagcgtgtttggcgtcatgtctagcacgctctacacgatcccgttcaacctaattgctgagtatcagcgggaggaagaggaacaagtaaagctggagggaggcaatgaaagccctcgaggcaccgggatggactgtgctgcgctcacttgtatggtccagctggctcaaatcattgtgggggcgggactcggggctctggtcaacgcggcaggaagtgtcatcgtggtggttctatcagcttcctctatctccctcataggctgcatcttcattgctctctttatcagatatgtggaatgatatttcttttaaagtgaactgtgctgctttattaaataaagagtctttaatatatcaaaaaaaaa

SEQ ID NO: 2 - protein, 576 aa, Oryzias latipes
MTLLSEDQSARPQPCLLSEPDKHISTSLDQYRPDYTKEDYMDSVETSVFGTVEPPRRSRGRLILHSMVMFGREFCYAVEAAFVTPVLLSVGLPRSLYSLVWLISPILGFLLQPIIGSASDYCRSSWGRRRPYILVLGILMLVGLSMFLNGDAVVSELVSDRSSRSTWAIVVVMFGVVLFDFAADFIDGPIKAYLFDVCSYQDKERGLHYHALFTGLGGACGYLVGAMDWGHSVLGRLLGSEYQVIYFFSALTWGVFLIVHLFSIPEKPLAKVPSESSASSALRLLGPHSNGYGALGKEPVSPVIPTSSPEIRPRSYSALGERPRSFSALGEANSVTSSAKQPIKEDQKKMTFRSLMKAIFNMPNHYRFLCISHLLGWAAFLCNMLFFTDFMGQIVYRGNPYAEHNSTAYITYERGVEVGCWGLCINAVSSALYSYVQRFLLPYIGLKGLYFMGYFVFGMGTSLIGLFPEVIATLILCSVFGVMSSTLYTIPFNLIAEYQREEEEQVKLEGGNESPRGTGMDCAALTCMVQLAQIIVGAGLGALVNAAGSVIVVVLSASSISLIGCIFIALFIRYVE

SEQ ID NO: 3 - DNA, 1650 bp, Homo sapiens
  CDS (26)..(1615)
caggaaggttcctctcccagtggccatgggtagcaacagtgggcaggctggccgccacatctataaatccctagctgatgatggcccctttgactctgtggagccgcctaaaagacccaccagcagactcatcatgcacagcatggccatgttcggaagagagttctgctacgcggtggaggcagcgtatgtgaccccagtcctgctcagcgtaggtctgcccagcagcctgtacagcattgtgtggttcctcagccccatcctgggattcctgctgcagcccgtggtcggatcggccagcgaccactgccggtccaggtggggccgccggagaccctacatcctcaccctgggagtcatgatgctcgtgggcatggctctgtacctcaatggggctactgttgtagcagctttgattgctaacccaaggaggaagctggtttgggccataagtgtcaccatgataggtgtcgttctctttgattttgctgccgacttcattgatgggcccatcaaagcctacttatttgatgtctgctcccatcaggacaaggagaagggcctccactaccatgccctcttcacaggttttggaggtgccctgggttaccttttgggtgctatagactgggcccatctggagctgggaagactgttgggtacagaattccaggtcatgttcttcttctctgcattggtgctcactttgtgttttactgttcatctgtgcagtatctctgaagccccacttacagaggttgcaaagggcattcccccacagcaaacccctcaggaccctccattgtcatcagatggaatgtacgagtatggttctatcgagaaagttaaaaatggttacgtaaatccagagctggcaatgcagggagcaaaaaacaaaaatcatgctgaacagactcgcagggcaatgacattaaagtcactgctgagagcactggtgaacatgcctcctcactaccgctacctttgcatcagccacctcattggatggacggccttcctgtccaacatgctgttcttcacagatttcatgggccagattgtgtaccgcggggatccctatagtgcacacaactccacagagtttctcatctacgaaagaggagtcgaggttggatgttggggcttctgcatcaactccgtgttttcctcactttattcttactttcagaaagttttggtatcctacattggattaaagggtctttacttcacgggatatttgctgtttggcctggggacgggatttattgggctcttcccgaatgtctactccaccctggtcctgtgcagcctgtttggtgtaatgtccagcaccctgtacactgtgccctttaacctcattactgagtaccaccgcgaggaagaaaaggagaggcagcaggccccaggaggggacccagacaacagcgtgagagggaagggcatggactgcgccaccctcacatgcatggtgcagctggctcagatcctggtcggaggtggcctgggctttctggtcaacacagccgggaccgttgtcgtcgtggtgatcacagcgtctgcggtggcactgataggctgttgctttgtcgctctctttgttagatatgtggattaggtcaataaagagacaatgaccctaaaaaaaaa

SEQ ID NO: 4 - protein, 530 aa, Homo sapiens
MGSNSGQAGRHIYKSLADDGPFDSVEPPKRPTSRLIMHSMAMFGREFCYAVEAAYVTPVLLSVGLPSSLYSIVWFLSPILGFLLQPVVGSASDHCRSRWGRRRPYILTLGVMMLVGMALYLNGATVVAALIANPRRKLVWAISVTMIGVVLFDFAADFIDGPIKAYLFDVCSHQDKEKGLHYHALFTGFGGALGYLLGAIDWAHLELGRLLGTEFQVMFFFSALVLTLCFTVHLCSISEAPLTEVAKGIPPQQTPQDPPLSSDGMYEYGSIEKVKNGYVNPELAMQGAKNKNHAEQTRRAMTLKSLLRALVNMPPHYRYLCISHLIGWTAFLSNMLFFTDFMGQIVYRGDPYSAHNSTEFLIYERGVEVGCWGFCINSVFSSLYSYFQKVLVSYIGLKGLYFTGYLLFGLGTGFIGLFPNVYSTLVLCSLFGVMSSTLYTVPFNLITEYHREEEKERQQAPGGDPDNSVRGKGMDCATLTCMVQLAQILVGGGLGFLVNTAGTVVVVVITASAVALIGCCFVALFVRYVD

SEQ ID NO: 5 - DNA, 1986 bp, mouse
  CDS (5)..(1594)
ggtcatgagtggaagcaatgggccgactgacacccatacctatcaatccttagccgaggattgcccctttggctctgtggagcaacccaagagatccacagggagacttgtcatgcacagcatggccatgtttggccgagagttttgctatgcggtggaggcagcttatgtgactccagttctgctcagcgtgggcctgcctaagagcctgtacagcatggtgtggctcctaagccccatcttgggattcctgctccagcctgtggtgggatcagccagtgatcactgcagggcccgttggggtcgccggagaccatacatcctgactctggccattatgatgctcttgggaatggctctgtacctcaatggagatgcggtcgtatcagctttggttgctaacccaaggcagaagctgatctgggccataagcatcaccatggtaggtgtggttctcttcgatttttctgctgacttcattgacgggcccatcaaagcctacttatttgatgtctgctcccaccaggacaaggagaagggcctccactaccatgccctcttcacaggttttggaggtgcccttggctacattttgggtgccatagactgggtgcatctagatctgggaaggctgctgggcacagaattccaggtcatgttcttcttctctgccctggttctcatcttgtgcttcatcacacacctgtgcagtatccctgaagctccactcagagatgctgcaactgaccctccctcacagcaggaccctcagggctcgtcgctgtcagccagtgggatgcatgaatacggttctattgagaaagttaaaaatggaggtgcagacacagagcagccagtacaggaatggaaaaacaaaaagccttctggccagagtcagaggacaatgtcgatgaagtcactccttcgggcattagtaaacatgccttcccattatcgctgcctttgcgtcagccacctgattggatggactgccttcctgtcaaacatgctcttcttcacagatttcatgggacagattgtataccatggggatccctacggtgcacacaactccacggagtttctcatctatgaaagaggagttgaggtcggatgttggggcttgtgcatcaactctgtgttttcttcagtttattcatactttcagaaagctatggtctcctacattggattaaaaggcctttatttcatgggatatttgctctttggcctgggaacaggattcataggactctttccaaatgtgtactctactctggtcctctgttctatgtttggtgtaatgtccagcacattgtacactgtgccctttaacctcattgctgagtaccaccgtgaagaggagaaagagaaggggcaggaagcgccaggaggccctgacaaccaggggagaggcaaaggcgtggactgcgctgctctcacctgcatggtacagctggctcagatcttggttggaggtggcctgggcttcctggtcaacatggctgggagtgtcgtcgtcgtggtgatcacagcgtctgcggtgtccctgataggctgttgctttgtggcgctcttcgtaagatatgtagattaggttcataaaaagacacccaccaaacctcagwcaaaagaagcccatggcaagaaaccgctcttttctcggactcttcggtgttgttggggactgtatgcctcatgctggcactgcgccttgcacatacatgtggggaaggtgtggtgggattgcccgctcccctgcatcttccttactgccccgcaatgcaggatgagagagtcaagttctgcttctcaagagaatttcaagcctccatcatgtcttaggaacaaatgcatcttacytgtaatgcttataaataatttttaaaagtcaattcatataccactgggaaatcacacatttcatcaaattcagaagcacttaaaacatgaccgagtgttttttttaaaaaaaaaaaaaaaaaa

SEQ ID NO: 6 - protein, 530 aa, mouse
MSGSNGPTDTHTYQSLAEDCPFGSVEQPKRSTGRLVMHSMAMFGREFCYAVEAAYVTPVLLSVGLPKSLYSMVWLLSPILGFLLQPVVGSASDHCRARWGRRRPYILTLAIMMLLGMALYLNGDAVVSALVANPRQKLIWAISITMVGVVLFDFSADFIDGPIKAYLFDVCSHQDKEKGLHYHALFTGFGGALGYILGAIDWVHLDLGRLLGTEFQVMFFFSALVLILCFITHLCSIPEAPLRDAATDPPSQQDPQGSSLSASGMHEYGSIEKVKNGGADTEQPVQEWKNKKPSGQSQRTMSMKSLLRALVNMPSHYRCLCVSHLIGWTAFLSNMLFFTDFMGQIVYHGDPYGAHNSTEFLIYERGVEVGCWGLCINSVFSSVYSYFQKAMVSYIGLKGLYFMGYLLFGLGTGFIGLFPNVYSTLVLCSMFGVMSSTLYTVPFNLIAEYHREEEKEKGQEAPGGPDNQGRGKGVDCAALTCMVQLAQILVGGGLGFLVNMAGSVVVVVITASAVSLIGCCFVALFVRYVD

(19) United States
(12) Patent Application Publication
(10) Pub. No.: US 2003/0175962 A1
(43) Pub. Date: Sep. 18, 2003

(54) Gene encoding b protein

(76) Inventors: Shoji Fukamachi, Chiba (JP); Akihiro Shima, Tokyo (JP); Atsuko Shimada, Tokyo (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/168,017

(22) PCT Filed: Dec. 17, 2001
(86) PCT No.: PCT/IB01/02518

(30) Foreign Application Priority Data
Jun. 8, 2001 (JP) 2001-174473

(51) Int. Cl. C12N 5/00; C07H 21/04

(52) U.S. Cl. 435/375; 536/23.2

(57) ABSTRACT

An objective of this invention is to identify and provide a gene encoding medaka protein B. An additional objective of the present invention is to provide human and murine homologues of the gene. To achieve the above mentioned objectives, the inventors performed positional cloning on the B gene. The gene encoding medaka protein B was successfully isolated, and the present invention has come to completion. Accordingly, in accordance with the invention, there are provided a nucleic acid encoding medaka protein B and those encoding homologues thereof. According to another aspect of the invention, there are provided a nucleic acid listed in Seq. No. 1 that encodes medaka protein B and a nucleic acid listed in Seq. No. 3 that encodes mouse protein B/AIM-1. According to the invention, there is provided a method of suppressing melanin production characterized in that expression of the gene encoding protein B is suppressed.

### TECHNICAL FIELD

The present invention relates to genes essential for melanin synthesis, particularly to a gene encoding protein B essential for melanin synthesis.

Throughout the following description, various publications will be referred to by numerical citation within parentheses. Full bibliographic citation of these publications will be found immediately before the sequence listing at the end of the Detailed Description.

### BACKGROUND ART

For skin whitening as well as for prevention and treatment of skin spots and birthmarks associated with the formation of melanin pigment, conventionally, tyrosinase inhibitors have been used. However, there is still a strong demand for more efficacious medicaments and cosmetics that can be used safely for human as skin-whitening agents and therapeutic agents for skin spots and birthmarks. Accordingly, a method of suppressing melanin synthesis has been eagerly anticipated which is distinct from conventional ones.

Although melanin is known to be produced, in melanosomes within melanocytes, through tyrosinase-catalyzed and auto-oxidation of tyrosine, the mechanism of melanin production remains mostly unsolved, and most of the genes involved in melanin production have not been identified yet.

On the other hand, researches on vertebrate hair color have been pursued using primarily mouse as a model animal, and, during 1990s, a number of genes have been identified that are responsible for hair color mutation. These researches have not only made a great contribution to the elucidation of pigment cell differentiation at molecular level, also revealed their association with human genetic diseases (1-6). However, mammals, during the course of evolution, culminated in holding decreased kinds of pigment cells and losing mobility of pigment granules in cells. Therefore, it will be impossible to elucidate, by researches using mouse, the molecular mechanisms underlying the features recognized in lower vertebrates, namely, bright body coloration, marking formation, and rapid body color change for adaptive response to environment. In contrast, medaka (rice fish; Oryzias latipes) is thought to be equally qualified to zebrafish as a model organism of vertebrates, and recently bases for its molecular genetic researches have been rapidly being set (7, 8). Since medaka has all sets of pigment cells (black and yellow/red, silver, and white), and approximately 70 kinds of spontaneous mutants in body color have been separated and established as strains, it is thought to be very useful as a model organism for body color research (9). A medaka with orange color is called himedaka, which has been a popular companion animal in Japan from a long time ago. While xanthophore/erythrophore on body surface are normal in himedaka, the amount of melanin in its melanophore is extremely small. It is known that this phenotype is due to mutation of a single gene (B gene) located in a b locus. However, the B gene remains unidentified and its function is only presumptive.

### DISCLOSURE OF INVENTION

Accordingly, an objective of the present invention is to identify and provide a gene encoding medaka protein B. An additional objective of the present invention is to provide human and murine homologues of the gene.

This invention also relates to a method of suppressing melanin production characterized in that expression of a nucleic acid(s) encoding medaka protein B or a homologue(s) thereof is(are) suppressed.

To achieve the above mentioned objectives, the inventors performed positional cloning on the B gene. The gene encoding medaka protein B was successfully isolated, and the present invention has come to completion.

Accordingly, in accordance with the invention, there are provided a nucleic acid encoding medaka protein B and those encoding homologues thereof.

According to another aspect of the invention, there are provided a nucleic acid listed in Seq. No. 1 that encodes medaka protein B and a nucleic acid listed in Seq. No. 3 that encodes mouse protein B/AIM-1.

In this case, the nucleic acids may be DNA, more preferably be cDNA or genomic DNA.

According to the invention, there is provided a method of suppressing melanin production characterized in that expression of the gene encoding protein B is suppressed.

### BRIEF DESCRIPTION OF DRAWINGS

FIG. 1 is a photograph showing the difference between phenotypes of wild-type and b-locus mutants. From top to bottom: wild-type (B/B), b (b/b) and b^{g8} (b^{g8}/b^{g8})

FIG. 2 is a genetic and physical map of the b mutation candidate interval.

a, The recombination map of linkage group 12 (LG12).

b, Further recombination analysis with another 250 female meioses did not detect recombination between the b locus and OPH3-1. Terminal sequences of OPH3-1-positive BAC (171M23) were mapped and named 171M23F and 171M27R.

c, BAC171M23 was subcloned into cosmids and several OPH3-1-positive clones were isolated by PCR screening. Shown is one cosmid (C27) with an insert of about 40 kb, which was shown to contain the entire b-mutation candidate.

d, Shotgun sequencing determined 36.3 kb (>90%) of the C27 insert.

FIG. 3 shows medaka B cDNA and the deduced amino-acid sequence.

a, Nucleotide sequence of B and the amino-acid sequence deduced from its cDNA.

b, Sequence alignment of medaka protein B, human AIM-1 protein, and the mouse B/AIM-1 homologue.

FIG. 4 illustrates an experiment to investigate mutations of the B gene.

a, A gel-electrophoretic photograph after RT-PCR performed in two regions of B mRNA using pairs of primers indicated above.

b, Model for the arrangement of the B polypeptide in the membrane.

FIG. 5 illustrates an experiment to investigate expression of the B gene.

a, A gel-electrophoretic photograph after RT-PCR was performed independently on two regions of B mRNA using a primer pair indicated at the top of the figure, and then expression was continuously detected from the day of fertilization (day 0) until hatching (day 7).

b, A microphotograph showing the in situ hybridization with the partial B cDNA riboprobes to a two-day embryo of the albino i6 strain which completely lacks melanin in the melanophores. Left: sense probe, right; antisense probe.

c, A microphotograph showing distribution of melanophores in a wild-type embryo.

d, A microphotograph showing expression of B in two-day embryos of the b strain. Left: sense probe, right; antisense probe.

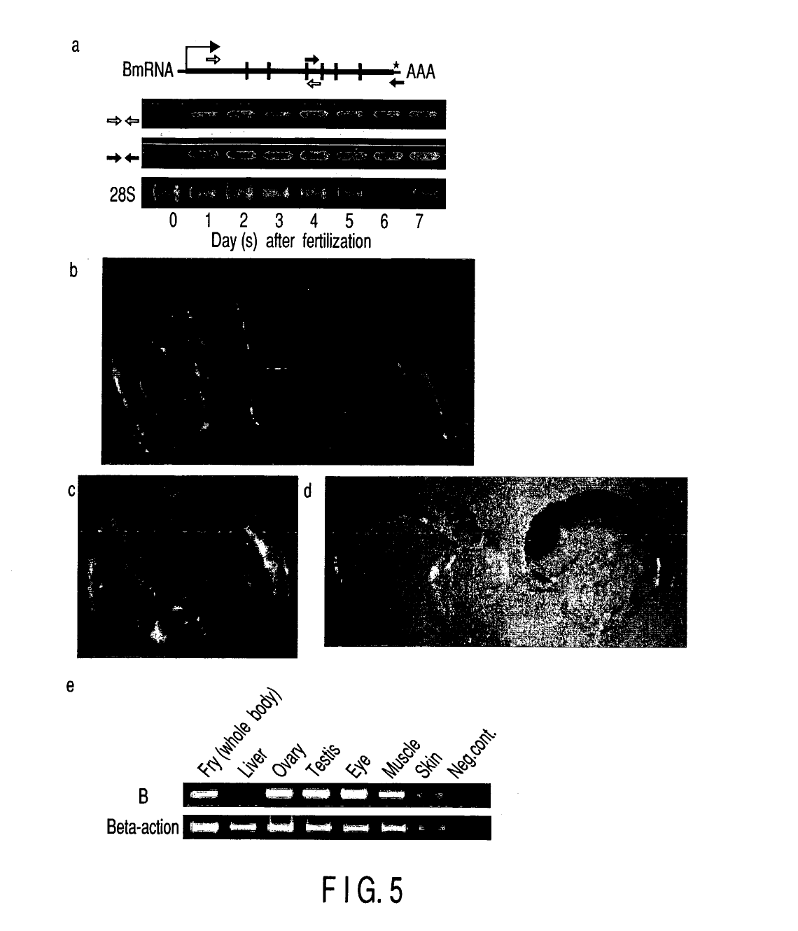

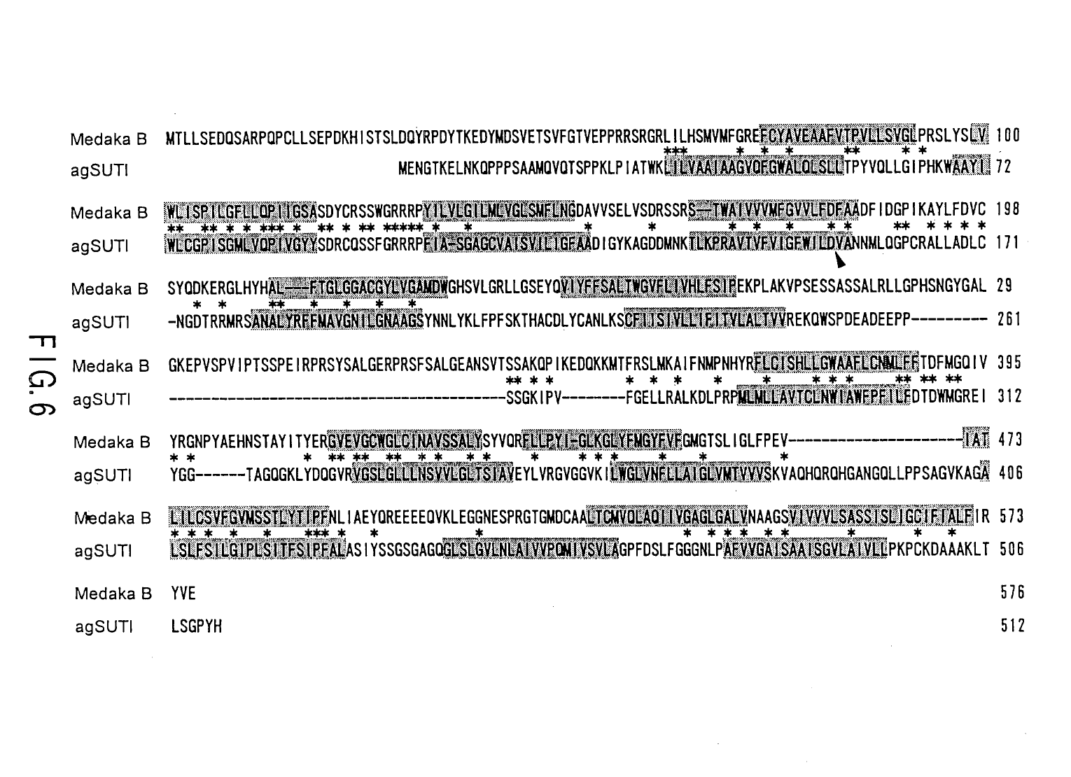

e, A gel-electrophoretic photograph showing B gene expression in adult organs. The primers indicated by black arrows in FIG. 5a were used.

In FIG. 6 is shown an amino acid sequence alignment of medaka protein B (HNI strain) and Apium graveolens sucrose transporter (agSUT1).

### BEST MODE FOR CARRYING OUT OF THE INVENTION

Hereafter, the invention will be illustrated in more detail.

The inventors have identified the medaka B gene using positional cloning.

The positional cloning is an approach in which the position of a responsible gene on a chromosome is defined with a variety of methods to carry on the cloning of the responsible gene with the aid of the defined position. The position and region of a target gene are determined by means of linkage analysis method using crossing. Molecular (DNA) markers are essential for these analyses. Physical mappings are made by constructing, with the use of chromosome walking using the above markers and BAC (bacterial artificial chromosome) and the like, a contig over a region where the target gene is located. Confirmation of a candidate gene will be achieved by capturing gene fragments expressed in the above regions to analyze DNA polymorphism between normal individuals and mutants. Screening of full-length cDNA clones thereafter, for example, will be performed conventionally with general cloning techniques using the above gene fragments as probes.

Specifically, the inventors isolated the B gene as a responsible gene for a mutant in b locus by means of linkage analysis using crossing between wild-type and himedaka strains, and chromosome walking using BAC. In this BAC DNA stage, the target region can be narrowed down to 200 Mb in size. Then techniques will be applied in which the cloned DNA in BAC is fragmented and subcloned into a vector using E. coli as a host. It is most preferable to subclone DNA fragments digested to 30-40 kb into a cosmid vector. Therefore, in this study, cosmid libraries were prepared from BAC containing the b locus. Screening was carried on to narrow the candidate region to less than 40 kb. As a result of sequencing an insert in a cosmid containing the candidate region for b mutation, a candidate gene having high homology with human MACR1 and AIM-1 was found. While neither mutation of nucleic acids nor expression of abnormal mRNAs was detected in medaka MACR1, a mutation was identified in AIM-1 homologous gene, and the inventors designated the gene as B.

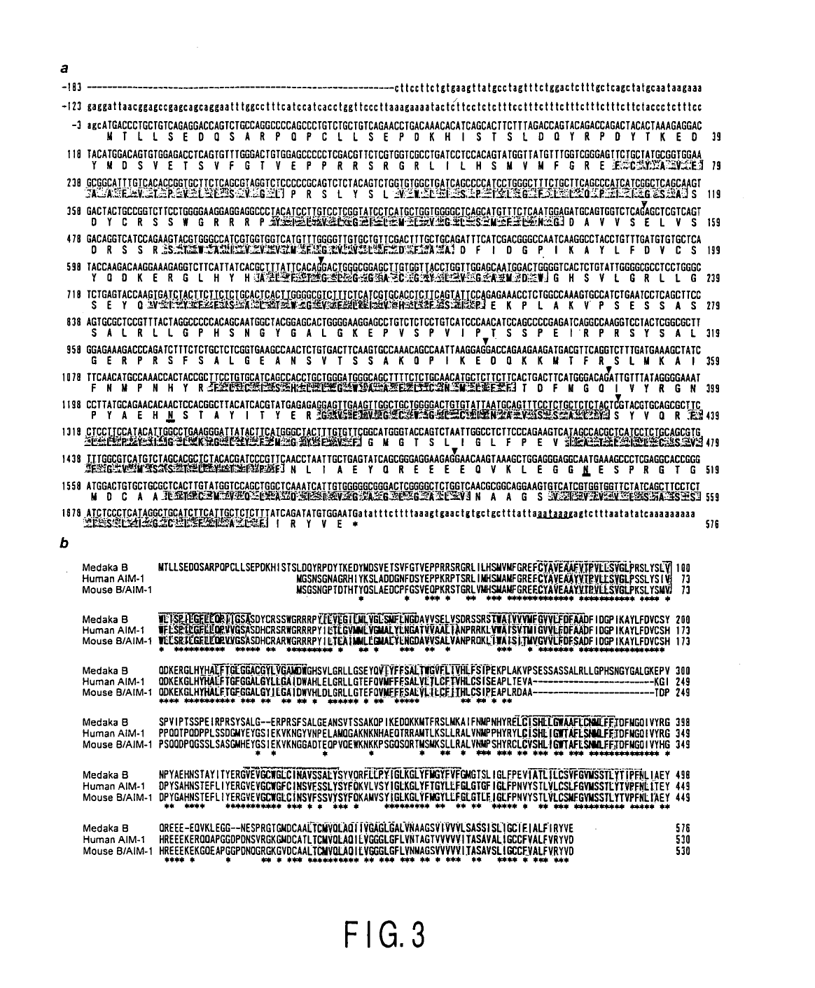

The nucleic acid sequence encoding medaka protein B (Seq. No. 4) is that listed in Seq. No. 1. A human homologue of medaka protein B encodes human AIM-1 (Seq. No. 5), which is reported as an EST isolated from melanoma cells. The nucleotide sequence encoding human AIM-1 is that listed in Seq. No. 2. The nucleotide sequence encoding a murine homologue of medaka protein B (Seq. No. 6) is that listed in Seq. No. 3 (FIG. 3). The murine homologue according to the invention was cloned with degenerated primers. Namely, cDNAs for cloning the murine homologue of the invention can be prepared by synthesizing single-stranded cDNAs, with the use of a commercial kit using mRNAs from murine cells as templates, by means of oligo dT primers and reverse transcriptase. In order to isolate the DNA of interest with the cDNAs prepared as described above, for example, degenerate primers are first synthesized that are corresponding to the amino acid sequence of medaka protein B, then degenerate polymerase chain reaction (also referred to as degenerate PCR) is carried out. Primers are then synthesized based on the resultant partial sequence from gene of interest, and 5′ and 3′ RACE are performed using the primers. “RACE” (rapid amplification of cDNA ends) herein mentioned is a method of running PCR based on nucleotide sequence information obtained from a known region, when the nucleotide sequence of cDNA is previously known in part, to clone an unknown region up to cDNA ends. When the unknown region is located upstream (5′) side of cDNA it is called 5′ RACE, and when located downstream (3′) it is called 3′ RACE. For example, in a case of 5′ RACE, antisense primers are synthesized on the basis of a known nucleotide sequence to make a reverse transcription reaction using the synthesized primers. Subsequent digestion of mRNA (forms a double strand with cDNA) with RNaseH results in single-stranded first-strand cDNA. While the first-strand cDNA contains a primer-derived sequence in its 5′ terminus, the 3′ terminus ends with an unknown sequence. Therefore, an anchor sequence is to be added to the 3′ terminus in order to amplify the unknown sequence by PCR. The cDNA having an unknown region in its 5′ upstream region can be amplified by running PCR, after the addition of the anchor sequence, using a nucleotide primer complementary to the anchor sequence and an antisense primer specific for a partial region whose sequence is known. In addition to cDNA, genomic DNA may be included as a template DNA used for degenerate PCR.

Nucleotide sequences of DNA fragments prepared with 5′ RACE and 3′ RACE are then determined. The nucleotide sequences can be determined by conventional methods such as chemical modification method of Maxam-Gilbert or dideoxynucleotide chain-termination method using M13 phage. Generally, automatic nucleotide sequencers (e.g., 373A DNA sequencer manufactured by PERKIN-ELMER Co. Ltd.) will be used for sequencing.

The function of the medaka protein B or homologues thereof described above has not been known so far, and they have never been reported to have similar characteristics to those found in the present invention. Also, it has not been reported that the genes of the present invention are essential for melanin synthesis.

The nucleic acids of the invention can be produced by the methods described above and those described in the Examples. Since their nucleotide sequences have already been determined, they can also be produced by chemical synthesis, by PCR using the genes as templates, or by hybridization using DNA fragments sharing the nucleotide sequences of the genes as probes. Designed primers or probes can be chemically synthesized according to their nucleotide sequences. PCR can be performed according to conventional procedures.

As used herein, the term “medaka protein B or homologues thereof” refers to full-length medaka protein B or homologues thereof, biologically active fragments thereof, or functional equivalents of the medaka protein B or homologues thereof. As functional equivalents of the medaka protein B or homologues thereof, modified medaka protein Bs or homologues thereof having any biological activity may be included. Functional equivalents of the medaka protein B or homologues thereof may include, for example, those proteins wherein one or several amino acid(s) is (are) replaced for (e.g. replacement of one acidic amino acid with a different acidic amino acid), deleted from, or added to their amino acid sequences.

The nucleic acids of the invention encoding the medaka protein B or homologues thereof may encode either whole of these proteins or shorter peptide sequences that can have an effect on melanin synthesis. Alternatively, said nucleic acids may have nucleic acid sequences that encode, due to degeneracy in genetic codes, the gene products identical to the medaka protein B or homologues thereof.

As an example of the nucleic acid sequences of the invention encoding the medaka protein B or homologues thereof, the followings can beincluded: DNA sequences encoding the medaka protein B or homologues thereof, and RNAs encoded by the DNA sequences.

As used herein, the term “homologues of the medaka protein B” means proteins having homology to the medaka protein B. More specifically, they share preferably 45% or more, more preferably 60% or more, more preferably 75% or more, even more preferably 90% or more, and most preferably 95% or more sequence homology with the deduced amino acid sequence of the medaka protein B.

In order to search the homologues described above and their homology, sequences available in public databases can be used for searching with the aid of program (e.g., Blastn/Blastx25, but not limited to these).

By comparison with the amino acid sequences described above, the medaka protein B will be found to share 55% amino acid identity with human AIM-1 (see FIG. 3).

General procedures can be used to suppress the expression of nucleic acids. For example, antisense method may be used. In this method, antisense RNA is injected exogenously into adult animals or cells; wherein the antisense RNA is such RNA containing a sequence complementary to whole or part of an mRNA nucleotide sequence. The RNA so injected inhibits, through the formation of a hybrid with the mRNA, a process of translation of genetic information carried by mRNA into protein. Said antisense RNA can be expressed within the cells as antisense RNA by incorporating its DNA information into an expression vector. Since they both are structurally complementary to target mRNA, they can bind to the target mRNA to suppress the expression of target gene. For exhibiting antisense effect, the antisense RNA may not be 100% complementary to the target RNA. The complementarity may not be high so long as the expression of the protein of the invention can be suppressed. The antisense RNA shares preferably 90%, more preferably 95% complementarity with the RNA of the invention. For exhibiting antisense effect, the length of the complementary RNA must be at least 15 bp, preferably 100 bp or more, more preferably 500 bp or more. We can also use antisense DNA complementary to the DNA encoding the medaka protein B or a part thereof. The antisense RNA complementary to the DNA of the invention can be prepared according to general procedures.

### EXAMPLES

This invention will be illustrated in more detail with reference to the following Examples. However, one skilled in the art will readily appreciate that these Examples are merely illustrative of the invention and should not be intrigued as limiting the scopes of the invention.

Methods

Linkage Analysis of Medaka;

Linkage analysis was performed using backcross progeny from genetically divergent HNI (+/+) (12) and AA2 (b/b) (13) inbred strains. The BAC genomic library used for screening is based on the Hd-rR (b/b) inbred strain (8).

Genomics;

BAC and cosmid ends were sequenced directly after purifying BAC or cosmid DNA using the Plasmid Maxi Kit (Qiagen), and mapped as described elsewhere (11). BAC171M23 was subcloned into the SuperCos 1 Cosmid Vector after Sau3AI partial digestion, followed by phage packaging using MaxPlax Lambda Packaging Extract (Epicenter Technologies). A shotgun library was constructed by subcloning Sau3AI-partially-digested C27 fragments into BamHI site of pUC118. Inserts were amplified by colony PCR and sequenced on the Applied Biosystems model 377 automated DNA sequencer. Sequence data were manually assembled using Genetixmac. The C27 sequence was compared with sequences that are publicly available in databases, using the Blastn and Blastx (25) programs.

Mutation Analyses;

First-strand cDNA was prepared from total RNA isolated from hatched fries using Isogen (Nippon Gene). RT-PCR of the b alleles was performed in three or more overlapping regions using the following parameters: 30 cycles of 98° C. for 20s, 60° C. for 1 min, and 72° C. for 2 min, and products were sequenced directly.

Mouse B/AIM-1 Homologue;

Nested degenerate primers were designed from medaka B and human AIM-1 sequences, and mouse cDNA from adult (albino) eyes, kidneys, and uterus were used as templates for RT-PCR. To determine the whole sequence of the ORF, specific primers were designed to the amplified sequence for 3′-RACE and 5′-RACE.

Expression;

Fertilized eggs of the Sakura strain (+/+) were incubated at 25° C. and killed 0-7 days after fertilization. To assess adult expression, the HNI strain was used. RT-PCR products were separated electrophoretically on 1% agarose gel and examined under UV-transillumination after ethidium bromide staining. Whole mount in situ hybridization was performed by hybridizing DIG-labelled partial B riboprobes to tyrosinase deletion mutant (i6/i6) (26) embryos.

Results

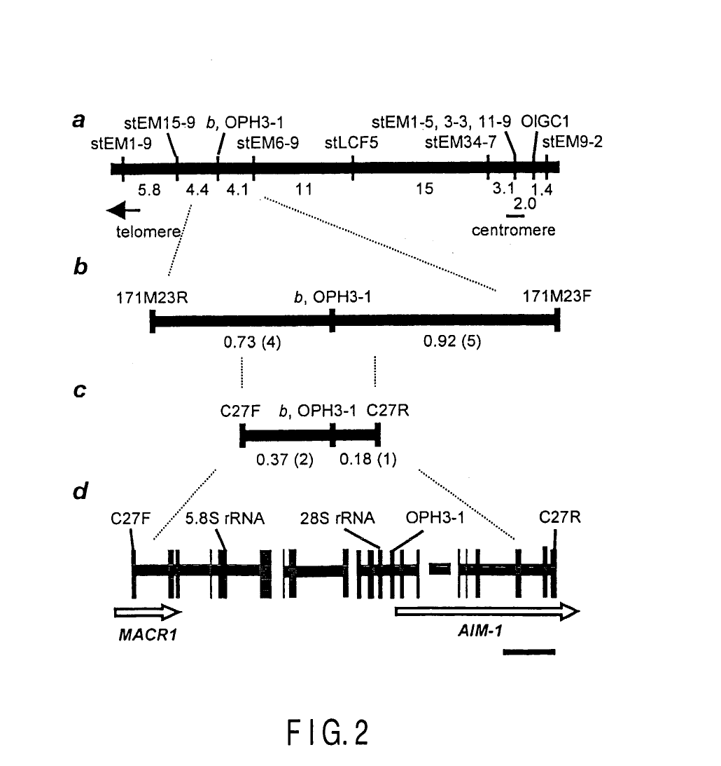

We have previously reported an STS (called OPH3-1) that is tightly linked to the b locus (11) (FIG. 2a). Further recombination analysis mapped the b locus to within 0-0.7 cM (0/545) of OPH3-1. Because the two inbred strains used for recombination mapping, HNI (12) (+/+) and AA2 (13) (b/b), are extremely polymorphic, it is not difficult to detect SNPs and/or other polymorphisms in every 100 bp of genomic sequence. To take advantage of this, we detected polymorphisms in terminal sequences of OPH3-1-positive BACs and mapped them using a PCR/PCR-RFLP method. Because an unexpectedly high level of recombination was detected (nine recombination within one BAC) (FIG. 2b), we further subcloned the BAC (171M23) into cosmids and isolated a cosmid (C27) that contains the entire b-mutation candidate region (FIG. 2c).

In this study, the extreme polymorphism and high recombination frequency enabled us to narrow the b-mutation candidate region to less than 40 kb by analyzing only 545 backcross progeny. This intriguingly high recombination frequency around the b locus is a female-meiosis-specific phenomenon, and it is about 10 times less frequent in males.

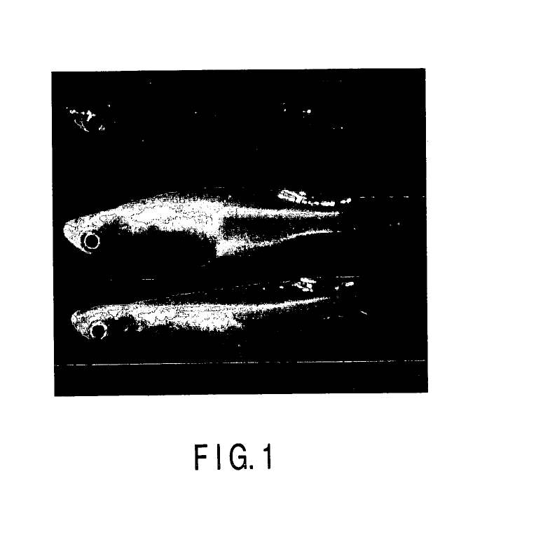

The BAC library screened was based on the Hd-rR (b/b) inbred strain (8). However, traces of B sequence were likely to exist in C27, because b is not a phenotypically null allele (FIG. 1). We actually obtained two B-candidate genes from 36.3 kb (>90%) of the sequence of the C27 insert (FIG. 2d). These were highly homologous to human genes encoding alpha-methylacyl-CoA racemase (MACR1) and AIM-1 protein. MACR1 catalyses the interconversion of R- and S-stereoisomers of alpha-methyl-branched-chain fatty acyl-CoA esters, and mutations in it cause adult-onset sensory motor neuropathy (14). AIM-1 has been reported as an EST isolated from melanocytes and melanoma cells and, more recently, as a new antigen on HLA-A2-expressing melanoma cells recognized by T cells (15).

Although neither nucleotide mutations nor abnormal mRNA expression was detected for medaka MACR1, we identified mutations on medaka AIM-1 in seven of eight b-locus mutants examined and named the gene B.

The whole B ORF was sequenced by 3′-RACE and its exon-intron boundaries determined (FIG. 3a). The deduced B protein consists of 576 amino acids and is 55% identical (304/543) to human AIM-1, although its N-terminus and middle regions are less conserved (FIG. 3b). Hydropathy analysis indicated that both medaka protein B and human AIM-1, and the mouse B/AIM-1 homologue which we isolated by degenerate RT-PCR, have 12 hydrophobic transmembrane domains and a less-conserved long loop between the sixth and seventh transmembrane domains (FIG. 3b).

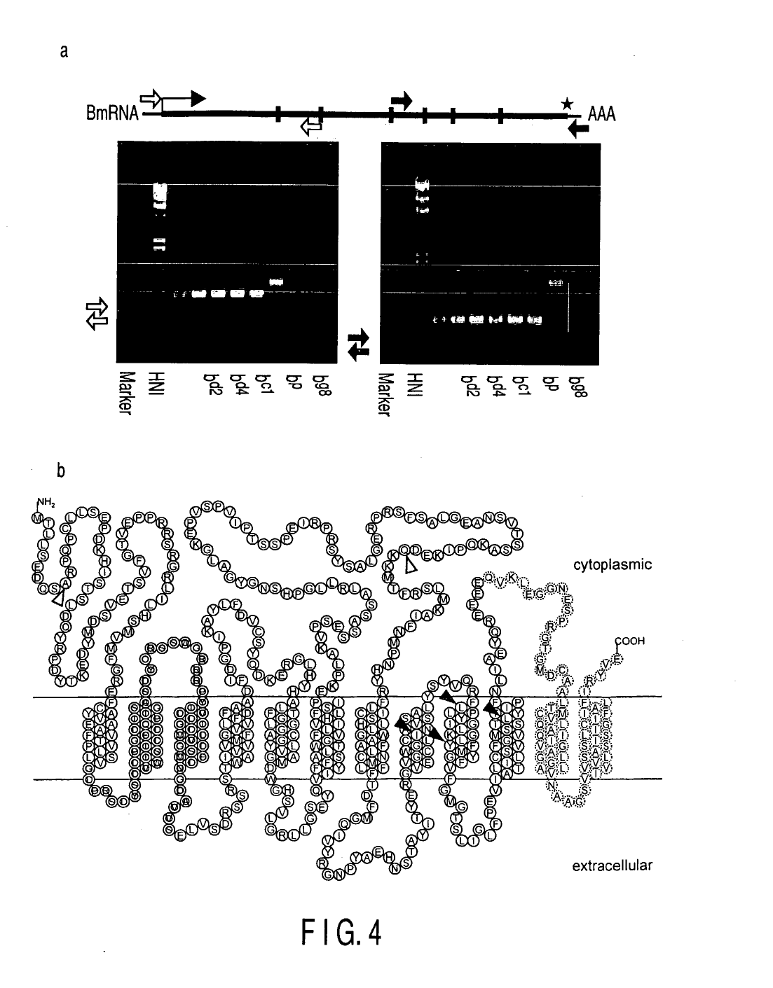

We analyzed two spontaneous (b and b^{p}), one radiation-induced (b^{g8}), and five ENU-induced (b^{g21}, b^{d2}, b^{d4}, b^{d8}, and b^{c1}) mutants. Abnormally longer RT-PCR products were amplified from b^{p} and b^{g8} (FIG. 4a). The b^{p} transcript has an insertion of 195 nucleotides, which is identical to the adjacent ORF sequence. This tandem repeat produces an insertion of 65 amino acids, which disrupts the normal conformation and function of B. The b^{g8} transcript has an insert of 699 nucleotides due to a missplicing of the last intron caused by a two-base deletion at the splice-donor consensus sequence. A premature stop codon in the intron causes truncation of B protein and loss of the eleventh and twelfth transmembrane domains. The b^{c1} transcript is normal in size but has a nonsense point mutation changing GAG (glutamine) to TAG, resulting in the loss of six C-terminus transmembrane domains. These results are consistent with phenotypic observation that these three more severe mutations result in less-melanized melanophores than do the b^{g21}, b^{d4}, b^{d2}, and b^{d8} missense point mutations that introduce charged hydrophilic amino acids into the transmembrane domains (FIG. 4b). In the b allele, the mutation does not occur in the ORF. However, our recombination map clearly shows that the b mutation is within the region covered by C27 that contains the first 1035 bp of the B ORF (FIG. 2c and d). Because b is distinguished from the other alleles in manifesting tissue-specific defects (FIG. 1) and B gene expression is disrupted in the b in

a tissue-specific manner, the b mutation may be in a gene-regulating region contained in C27:

a promoter, an enhancer, the 5′-UTR, or an intron.

The B gene is continuously expressed throughout embryonic development at levels detectable by RT-PCR (FIG. 5a). B transcripts detected at the blastula stage (day0) may be maternal. Strong expression signals were observed on the eyes, body, and yolk sac of two-day embryos (FIG. 5b), where melanophores are distributed in wild-type embryos (FIG. 5c). The signals on the body and yolk sac disappeared as the embryos developed, but those on the eyes were detected continuously until hatching, indicating transient B expression in developing melanophores. Adult fish also express the B gene and its expression is not restricted to the melanophore lineage (FIG. 5e). On the other hand, expression of human AIM-1 was not detected in any tissue other than melanocytes and melanoma cells by northern hybridization (15). However, almost identical ESTs from the kidney and uterus are reported in the databases. Furthermore, we isolated the mouse B/AIM-1 homologue from the eyes as well as from the kidney and uterus by degenerate RT-PCR. Therefore, B/AIM-1 genes are likely to be expressed in several organs although their strongest expression is restricted to the melanophore lineage.

With identification of the medaka B gene, we can begin to understand how this gene affects vertebrate melanization. Because the b mutation defect is manifested inside the melanosome (10) and many twelve-transmembrane proteins have been known to function as transporters (16-18), we propose that the B protein may be a component of the melanosomal membrane, transporting certain substances required for melanin biosynthesis. In this regard, it is quite informative that B has <23% (115/490) amino-acid identity with and structural similarity to plant sucrose-proton symporters (19) (FIG. 6). In particular, the region between the second and third transmembrane domains has significant similarity, where a completely conserved putative motif appears in all plant sequences known to function as sucrose transporters (19), in medaka B, in human AIM-1 and its mouse homologue, and also in the Drosophila CG4484 gene product (29% identical to medaka B). Although an effect of sucrose on melanin synthesis has not been postulated, mouse B16 melanoma cells cultured in medium containing galactose have much higher tyrosinase activity and produce more melanin than cells in medium containing glucose (20-21). Furthermore, it was recently reported that mutations at tyrosinase N-glycosylation sites reduce tyrosinase activity (22), indicating an effect of certain saccharides on melanin biosynthesis. Detailed functional analysis of the B/AIM-1 protein may provide new insights into the biochemical regulation of melanin synthesis.

### Advantage of the Invention

Through the use of the gene according to the present invention, a method will be achieved for suppressing melanin production in a manner different from conventional methods.

### REFERENCES

1. Besmer, P. et al. A new acute transforming feline retrovirus and relationship of its oncogene v-kit with the protein kinase gene family. Nature 320, 415-421 (1986).

2. Epstein, D. J., Vekemans, M. & Gros, P. Splotch (Sp2H), a mutation affecting development of the mouse neural tube, shows a deletion within the paired homeodomain of Pax-3. Cell 67, 767-774 (1991).

3. Hosoda, K. et al. Targeted and natural (piebald lethal) mutations of endothelin-B receptor gene produce megacolon associated with spotted coat color in mice. Cell 79, 1267-1276 (1994).

4. Baynash, A. G. et al. Interaction of endothelin-3 with endothelin-B receptor is essential for development of epidermal melanocytes and enteric neurons. Cell 79, 1277-1285 (1994).

5. Southard-Smith, E. M. et al. Sox10 mutation disrupts neural crest development in Dom Hirschsprung mouse model. Nat. Genet. 18, 60-64 (1998).

6. Gardner, J. M. et al. The mouse pink-eyed dilution gene: association with human prader-willi and angelman syndromes. Science 257, 1121-1124 (1992).

7. Naruse, K. et al. A detailed linkage map of Medaka, Oryzias latipes: comparative genomics and genome evolution. Genetics 154, 1773-1784 (2000).

8. Matsuda, M. et al. Construction of a BAC library derived from the inbred Hd-rR strain of the teleost fish, Oryzias latipes. Genes Genet. Syst. 76, 61-63 (2001).

9. Tomita, H. et al. In ‘Biology of the Medaka.’ Egami, N., Yamagami, K., and Shima, A., Eds., Univ. of Tokyo Press pp. 111-128. (in Japanese) (1990).

10. Matsumoto, J. & Hirose, E. Deficiency of the gene B impairs differentiation of melanophores in the medaka fish, Oryzias latipes: fine structure studies. Pigment Cell Res. 6, 45-51 (1993).

11. Fukamachi, S., Shimada, A., Naruse, K. & Shima, A. Genomic analysis of •-ray-induced germ-cell mutations at the b locus recovered from the medaka specific-locus test. Mutat. Res. (in press)

12. Hyodo-Taguchi, Y. Establishment of inbred strains of the teleost, Oryzias latipes. Zool. Mag. (Tokyo) 89, 283-301 (1980) (in Japanese).

13. Shima, A., & Shimada, A. The Japanese medaka, Oryzias latipes, as a new model organism for studying environmental germ-cell mutagenesis. Environ. Health Perspect. 102, 33-35 (1994) (suppl.).

14. Ferdinandusse, S. et al. Mutations in the gene encoding peroxisomal •-methylacyl-CoA racemase cause adult-onset sensory motor neuropathy. Nat. Genet. 24, 188-191 (2000).

15. Harada, M., Li, F. Y., El-Gamil, M., Rosenberg, A. S. & Robbins, F. P. Use of an in vitro immunoselected tumor line to identify shared melanoma antigens recognized by HLA-A*0201-restricted T cells. Cancer Res. 61, 1089-1094 (2001).

16. Kilty, J. E. & Amara, S. G. Families of twelve transmembrane domain transporters. Curr. Opin. Biotechnol. 3, 675-682 (1992).

17. Andrews, N. C. The iron transporter DMT1. Int. J. Biochem. Cell Biol. 31, 991-994 (1999).

18. Mount, D. B. et al. The electroneutral cation-chloride cotransporters. J. Exp. Biol. 201, 2091-2102 (1998).

19. Lemoine, R. Sucrose transporters in plants: update on function and structure. Biochim. Biophys. Acta 1465, 246-262 (2000).

20. Nakayasu, M., Saeki, H., Tohda, H. & Oikawa, A. Effects of sugars on melanogenesis in cultured melanoma cells. J. Cell. Physiol. 92, 49-55 (1977).

21. Saeki, H. & Oikawa, A. Effects of pH and type of sugar in the medium on tyrosinase activity in cultured melanoma cells. J. Cell. Physiol. 94, 139-146 (1978).

22. Branza-Nichita, N. et al. Mutations at critical N-glycosylation sites reduce tyrosinase activity by altering folding and quality control. J. Biol. Chem. 275, 8169-8175 (2000).

23. Shimada, A. & Shima, A. Combination of genomic DNA fingerprinting into the medaka specific-locus test system for studying environmental germ-line mutagenesis. Mutat. Res. 399, 149-165 (1998).

24. Shima, A. & Shimada, A. Development of a possible nonmammalian test system for radiation-induced germ-cell mutagenesis using a fish, the Japanese medaka (Oryzias latipes). Proc. Natl. Acad. Sci. USA 88, 2545-2549 (1991).

25. Altschul, S. F., Gish, W., Miller, W., Myers, E. W. & Lipman, D. J. Basic local alignment search tool. J. Mol. Biol. 215, 403-410 (1990).

26. Koga, A., Wakamatsu, Y., Kurosawa, J. & Hori, H. Oculocutaneous albinism in the i6 mutant of the medaka fish is associated with a deletion in the tyrosinase gene. Pigment Cell Res. 12, 252-258 (1999).

27. Henrique, D., Adam, J., Myat, A., Chitnis, A., Lewis, J. & Ish-Horowicz, D. Expression of a Delta homologue in prospective neurons of the chick. Nature 375, 787-790 (1995).

28. Tusnády, G. E & Simon, I. Principles governing amino acid composition of integral membrane proteins: applications to topology prediction. J. Mol. Biol. 283, 489-506 (1998).

1. A nucleic acid encoding medaka protein B or homologues thereof.

2. A nucleic acid listed in Seq. No. 1 that encodes medaka protein B.

3. A nucleic acid listed in Seq. No. 3 that encodes mouse protein B/AIM-1.

4. A method of suppressing melanin production characterized in that expression of at least one nucleic acid according to any one of claims 1-3 is suppressed.
